(19) United States
(12) Patent Application Publication
(10) Pub. No.: US 2026/0078445 A1
(43) Pub. Date: Mar. 19, 2026

(54) De Novo Sequencing of DNA

(71) Applicant: DH Technologies Development Pte. Ltd., Singapore (SG)

(72) Inventors: Takashi BABA, Richmond Hill (CA); Kaoru KARASAWA, Tokyo (JP)

(21) Appl. No.: 19/135,348

(22) PCT Filed: Dec. 2, 2023
(86) PCT No.: PCT/IB2023/062157

§ 371 Date: Jun. 3, 2025

Related U.S. Application Data
(60) Provisional application No. 63/386,414, filed on Dec. 7, 2022.

(51) Int. Cl. C12Q 1/6872

(52) U.S. Cl. CPC C12Q 1/6872

(57) ABSTRACT

A first product ion mass spectrum of a nucleic acid analyzed using a CID method is received. Also, a second product ion mass spectrum of the nucleic acid analyzed using a radical-induced dissociation method is received. Peak m/z values of the first spectrum, peak m/z values of the second spectrum, and an m/z value of a precursor ion of the nucleic acid are converted to a single charge. A peak m/z value of the first spectrum is determined that differs from a peak m/z value of the second spectrum by a mass value of a structure within the nucleic acid the radical-induced dissociation method is known to not be able to dissociate and the CID method is known to be able to dissociate. The structure includes a phosphorus atom and an optionally substituted 5-membered ring containing an oxygen.

### RELATED APPLICATIONS

This application claims the benefit of U.S. Provisional Patent Application Ser. No. 63/386,414, filed on Dec. 7, 2022, the content of which is incorporated by reference herein in its entirety.

### INTRODUCTION

The teachings herein relate to a method for de novo sequencing a nucleic acid from two mass spectra produced by two different dissociation methods. More particularly the teachings herein relate to systems and methods for locating a nucleotide of a nucleic acid using two different dissociation methods during de novo sequencing.

The systems and methods herein can be performed in conjunction with a processor, controller, or computer system, such as the computer system of FIG. 1.

### De Novo Sequencing Using Mass Spectra

De novo sequencing as used herein is defined as the reconstruction of the sequence of biomolecules directly from one or more mass spectra without additional information. Additional information can include, but is not limited to, genomic information or pre-obtained database information.

Valérie Gabelica et al. Electron Photodetachment Dissociation of DNA Polyanions in a Quadrupole Ion Trap Mass Spectrometer. Anal. Chem. 2006, 78, 18, 6564-6572 (doi/10.1021/ac060753p), (hereinafter the “Gabelica Paper”) have described a method of de novo sequencing. This method uses both a spectrum produced from electron photodetachment dissociation (EPD) and a spectrum produced from collision-induced dissociation (CID).

Specifically, the Gabelica Paper describes a two-step method for de novo sequencing of oligonucleotides. In a first step, the EPD spectrum is used to distinguish the d and w ion series from the a· and z· ion series. This is due to “the simultaneous observation, with very few exceptions, of d/a· and w/a· pairs separated by 99 Da” in an EPD spectrum. “In a second step, the w series can be identified simply by comparison with normal CID on the even-electron oligonucleotide, where w ion series fragments are formed, but not d fragments.”

The Gabelica Paper, however, also describes that its method has some limitations. One of its limitations, when compared with electron detachment methods is that “the presence of guanines is essential for EPD to occur” at a certain wavelength. The Gabelica Paper concedes that electron detachment dissociation (EDD) efficiency, for example, “is less base-dependent.” The Gabelica Paper does not, however, describe how the limitations of its method can be overcome or how its method might be applied to other dissociation techniques, such as EDD.

As a result, additional systems and methods are needed for de novo sequencing using mass spectra from two or more dissociation methods that overcome the limitations of the Gabelica Paper and are applicable to other dissociation techniques as well as EPD.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that for singly charged species, the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers. CID, in-source fragmentation, blackbody infrared radiative dissociation and IRMPD are examples of thermal-dissociation methods in this description. Thermal-dissociation methods included herein are non-radical dissociation methods that do not involve the use of radical formation in the dissociation process

ExD can include, but is not limited to, electron-induced dissociation (EID), electron impact excitation in organics (EIEIO), electron capture dissociation (ECD), or electron transfer dissociation (ETD). Radical-induced dissociation methods, mentioned herein, include ExD, UVPD, electron detachment dissociation (EDD), plasma electron detachment dissociation (pEDD), and electron photodetachment dissociation (EPD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

A system, method, and computer program product are disclosed for locating a nucleotide during de novo sequencing of a nucleic acid.

In step (A) of the method, a first product ion mass spectrum of a nucleic acid analyzed using a thermal-dissociation method is received. Also, a second product ion mass spectrum of the nucleic acid analyzed using a radical-induced dissociation method is received.

In an optional step (B), peak m/z values of the first spectrum, peak m/z values of the second spectrum, and an m/z value of a precursor ion of the nucleic acid are converted to a single charge.

In the cases that the optional step (B) is not applied before de novo sequencing, original spectra are used.

In step (C), a peak m/z value of the first spectrum is determined that differs from a peak m/z value of the second spectrum by a mass difference of a structure within the nucleic acid in which the radical-induced dissociation method is known to not be able to dissociate and with which the thermal-dissociation method is known to be able to dissociate.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary product ion spectrum obtained from applying a resonant CID method to fragment a nucleic acid compound, in accordance with various embodiments.

FIG. 3 is an exemplary product ion spectrum obtained from applying a plasma EDD method to fragment the same nucleic acid compound from which FIG. 2 was obtained, in accordance with various embodiments.

FIG. 4 is an exemplary diagrams showing empirical formulas of a structures that includes a phosphorus atom and an optionally substituted 5-membered ring containing an oxygen on the ring, in accordance with various embodiments.

FIG. 5 is an exemplary diagram showing that the fragmentation of the same nucleic acid by CID produces a-B ion series fragments and EDD produces a· ion series fragments that differ in mass by a known m/z, in accordance with various embodiments.

FIG. 6 is an exemplary diagram showing that the fragmentation of the same nucleic acid by CID and EDD produces w ion series fragments that do not differ in mass, in accordance with various embodiments.

FIG. 7 is an exemplary plot of the peak list ordered by singly charged m/z value, including a virtual starting peak, a· fragment candidates, and a virtual ending peak, in accordance with various embodiments.

FIG. 8 is an exemplary diagram showing the nomenclature of the different ion series fragments for a DNA compound and their relation to CID and EDD, in accordance with various embodiments.

FIG. 9 is a schematic diagram of a system for locating a nucleotide of a nucleic acid during de novo sequencing, in accordance with various embodiments.

FIG. 10 is an exemplary flowchart showing a method for locating a nucleotide of a nucleic acid during de novo sequencing, in accordance with various embodiments utilizing the empirical formulas describes in FIG. 4.

FIG. 11 is a schematic diagram of a system that includes one or more distinct software modules and that performs a method for locating a nucleotide of a nucleic acid during de novo sequencing, in accordance with various embodiments.

FIG. 12 contains depictions of 2^{nd} and further generation structures of nucleic acids that can be detected using the within teachings.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

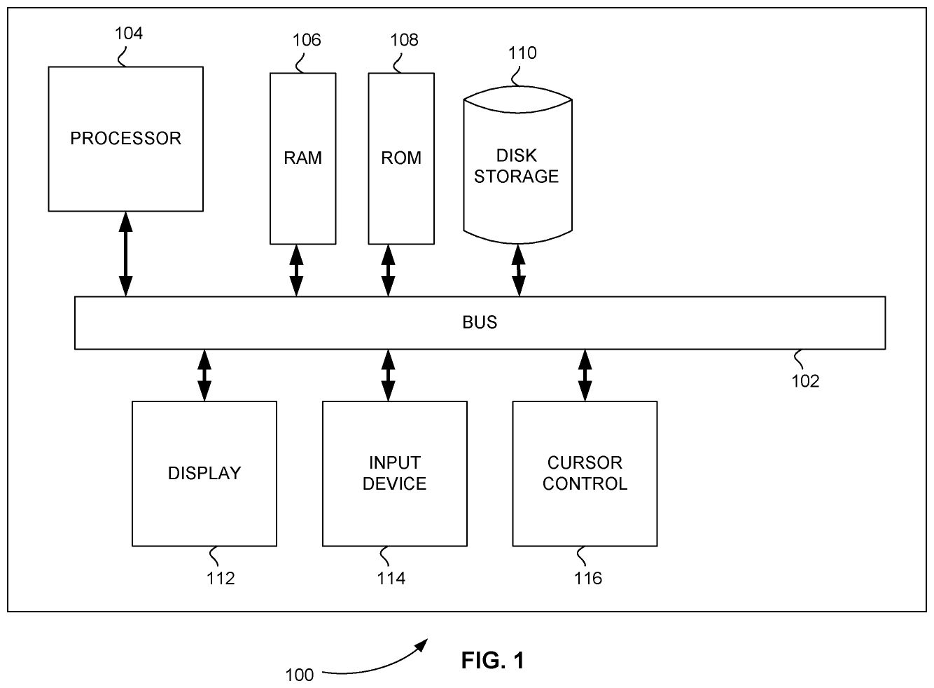

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product”are used interchangeably throughout this written description.

Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### De Novo Sequencing Using a·, a-B and w Ion Series Fragments

As described above, de novo sequencing is defined as the reconstruction of the sequence of biomolecules directly from one or more mass spectra without additional information. The Gabelica Paper describes a two-step method for de novo sequencing of oligonucleotides. In a first step, an EPD spectrum is used, and, in a second step, a CID spectrum is used.

The Gabelica Paper also describes that its method has a number of limitations. One of these limitations is the base-dependency of EPD. The Gabelica Paper does not, however, describe how these limitations can be overcome or how its method might be applied to other dissociation techniques, such as EDD.

As a result, additional systems and methods are needed for de novo sequencing that overcome the limitations of the Gabelica Paper and are applicable to other dissociation techniques as well as EPD.

In various embodiments, CID is performed as a thermal-dissociation method as examples. In alternative embodiments, IRMPD is performed as a thermal-dissociation method.

In various embodiments, EDD or pEDD is performed as a radical-induced dissociation method as examples.

In various embodiments, de novo sequencing of a nucleic acid compound is performed using a· and w ion series fragments of spectra obtained from two different dissociation techniques. The nucleic acid compound is a deoxyribonucleic acid (DNA) compound, for example. In various embodiments, the two different dissociation techniques comprise a thermal-dissociation technique and a radical-induced dissociation technique.

A number of steps are performed. First, a first spectrum from a thermal-dissociation method (e.g., CID method) and a second spectrum from a radical-induced dissociation method are obtained.

In a preferred embodiment, the thermal-dissociation method is a CID method and preferably is a resonant CID method and the radical-induced dissociation method is a plasma EDD method or a beam type negative ETD method. The use of resonant CID in analyzing DNA is described, for example, in U.S. Provisional Application No. 63/347,814, filed Jun. 1, 2022, which is incorporated herein by reference in its entirety. The use of plasma EDD in analyzing DNA is described, for example, in U.S. Provisional Application No. 63/347,808, filed Jun. 1, 2022, which is incorporated herein by reference in its entirety. The use of beam type negative ETD in analyzing DNA is described, for example, in U.S. Provisional Application No. 63/347,795, filed Jun. 1, 2022, which is incorporated herein by reference in its entirety.

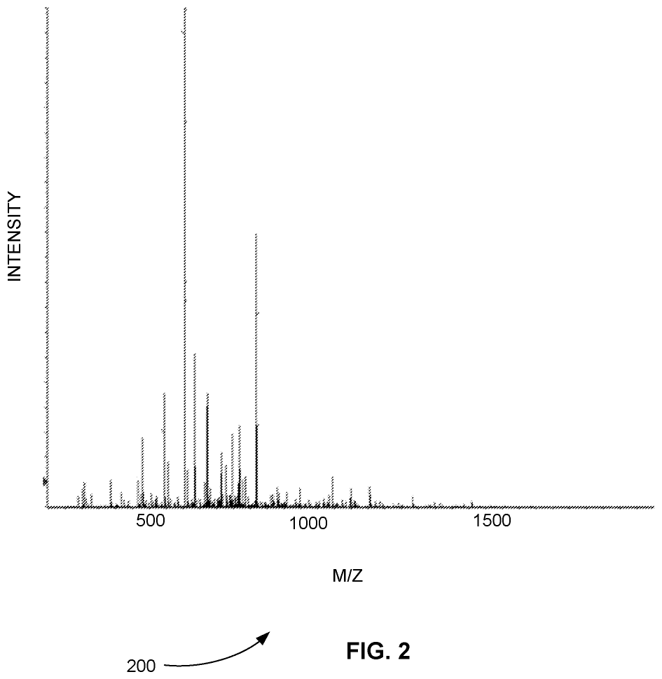

FIG. 2 is an exemplary product ion spectrum 200 obtained from applying a resonant CID method to fragment a nucleic acid compound, in accordance with various embodiments.

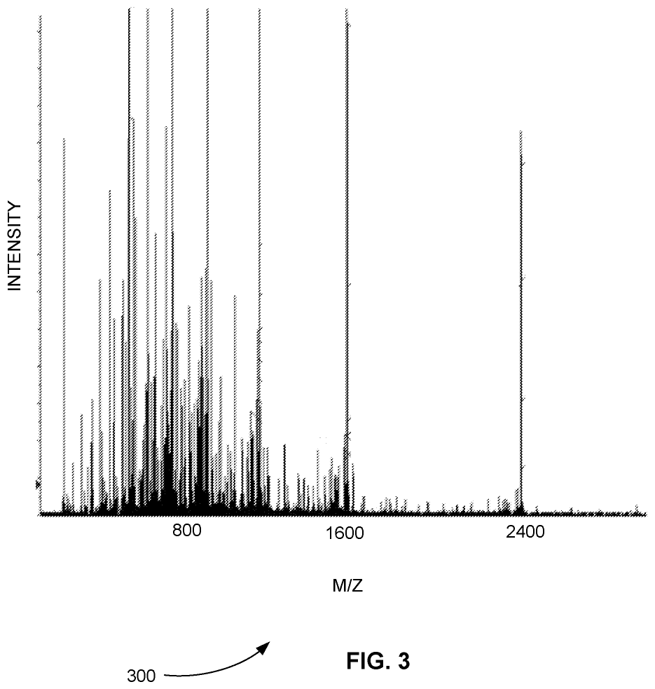

FIG. 3 is an exemplary product ion spectrum 300 obtained from applying a plasma EDD method to fragment the same nucleic acid compound from which FIG. 2 was obtained, in accordance with various embodiments.

In various embodiments, a radical-induced dissociation method can include, but is not limited to, any UVPD, EPD, ECD, ETD, EDD, pEDD, or electronic excitation dissociation (EED) method.

Next, in an optional step, the first spectrum and the second spectrum are converted to a single charge state. This is accomplished using charge state deconvolution, for example. In a preferred embodiment, the single charge state is z=−1.

Note that, in the following steps, the m/z values are absolute values (unsigned). An exception to this rule is the virtual starting peak of the de novo sequencing. This is a negative value, which is shown below.

Next, a starting or an ending m/z value for a nucleotide of a nucleic acid is found by finding a first peak from the first spectrum that differs from a second peak from the second spectrum by a mass difference of a structure within the nucleic acid the radical-induced dissociation method is known to not be able to dissociate and the thermal-dissociation method (such as CID) is known to be able to dissociate. In various embodiments, this structure includes a phosphorus atom and an optional substituted 5-membered ring containing an oxygen on the ring.

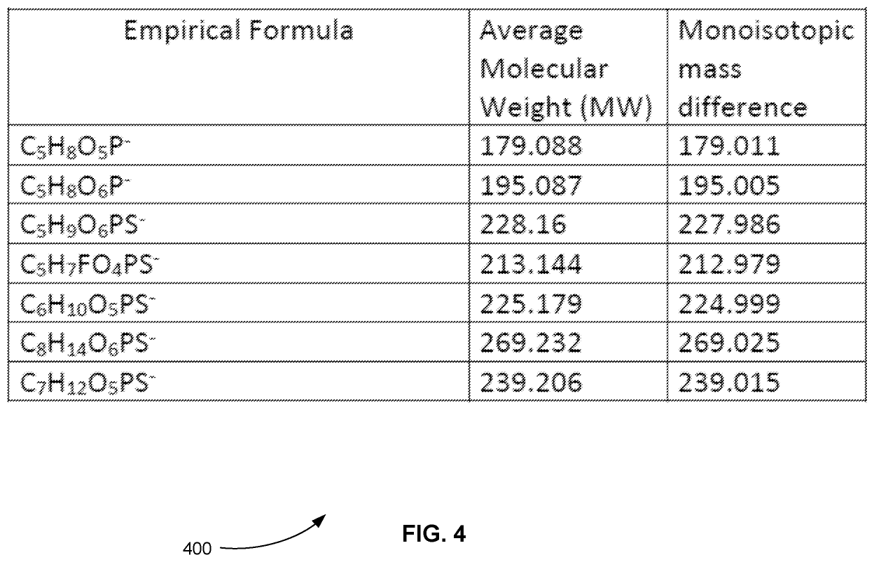

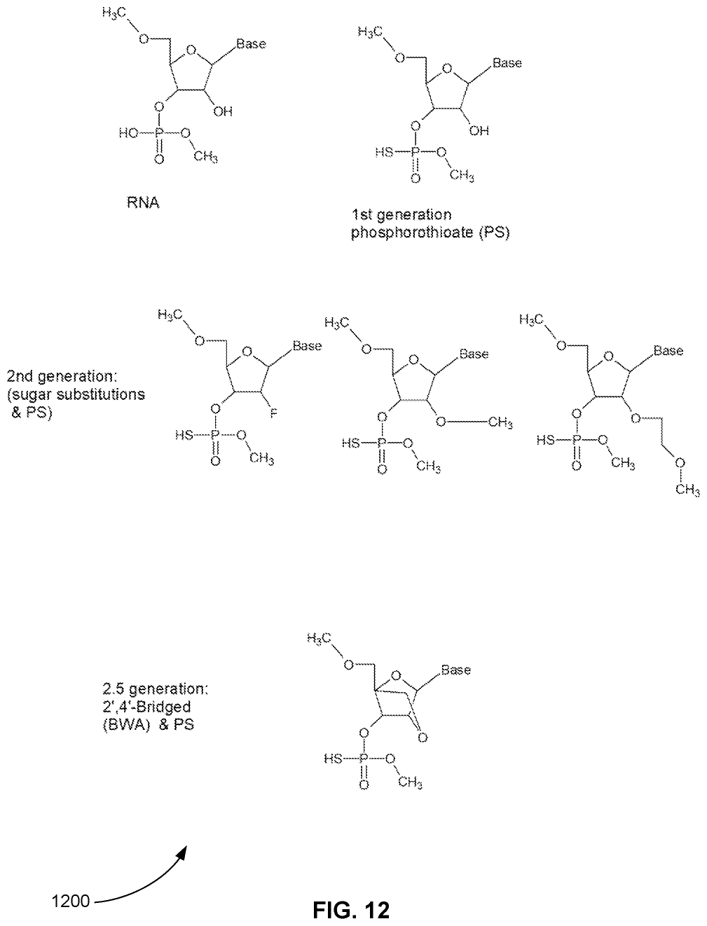

FIG. 4 is an exemplary diagram 400 showing a structural formula of a structure that includes a phosphorus atom and an optionally substituted 5-membered ring containing an oxygen, in accordance with various embodiments. In FIG. 4, the structure corresponds to one of the following empirical formulas: C₅H₈O₅P⁻, C₅H₈O₆P⁻, C₅H₉O₆PS⁻, C₅H₇FO₄PS⁻, C₆H₁₀O₅PS⁻, C₈H₁₄O₆PS⁻ or C₇H₁₂O₅PS⁻, along with associated monoisotopic differences that can be used in accordance with various teachings. These empirical formulas can be utilized to sequence units of modified oligonucleotides such as those depicted in FIG. 12. While the within teachings utilize masses with varying precisions with respect to decimal places, it should be noted that the three decimal places are preferred and can vary by +/−0.001 units.

In a preferred embodiment, this structure has the formula C₅H₈O₅P⁻ with a mass of 179.0115.

In various embodiments, all pairs of mass peaks with the mass difference of the compound in the single charge second (EDD) spectrum and the single charge first (CID) spectrum are found.

Again, in a preferred embodiment, all pairs of mass peaks with the mass difference of 179.0115 (C₅HO₅P) in the single charge second (EDD) spectrum and the single charge first (CID) spectrum are found. More precisely, when an m/z of a peak in the second (EDD) spectrum plus the a mass difference of 179.0115 of C₅H₈O₅P matches an m/z of a peak in the first (CID) spectrum, then the peak in the second (EDD) spectrum is listed as an a· ion series fragment candidate.

Note that one of ordinary skill in the art understands that when peaks are compared to a specific mass or m/z value the result of that comparison is within some predetermined threshold level, such as, but not limited to, the resolution of the instrument used to make the comparison.

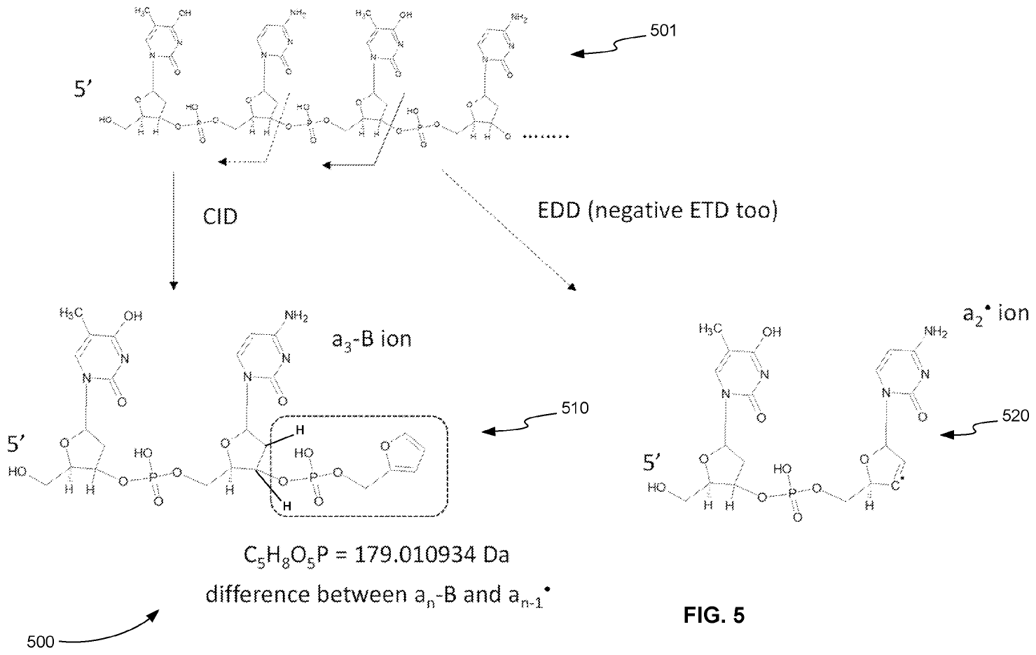

FIG. 5 is an exemplary diagram 500 showing that the fragmentation of the same nucleic acid by CID produces a-B ion series fragments and EDD produces a· ion series fragments that differ in mass by a known m/z (i.e., 179.0115 (C₅HO₅P)), in accordance with various embodiments.

FIG. 5 shows that the fragmentation of nucleic acid 501 by CID produces an a-B ion series fragment or product ion 510. However, the fragmentation of nucleic acid 501 by EDD produces a· ion series fragment or product ion 520. The difference in the mass between a-B ion series product ion 510 and a· ion series product ion 520 is 179.0115 Da, which is the known mass of C₅HO₅P⁻. This difference in fragmentation methods determines that a· ion series product ion 520 is an a· ion series fragment candidate.

The peak of the second (EDD) spectrum is listed as an a· ion series fragment candidate by placing the peak on a peak list ordered by m/z value.

Next, in an optional step, a singly charged precursor ion m/z value (z=−1) is calculated for the nucleic acid compound from the precursor ion m/z value and its charge state.

Next, all pairs with the mass difference value of 0 in the single charge second (EDD) spectrum and the single charge first (CID) spectrum are found. More precisely, when the m/z value of a peak in the second (EDD) spectrum is the same m/z as a peak in the first (CID) spectrum, the peak is listed as a w ion series fragment candidate.

Again, note that one of ordinary skill in the art understands that when peaks are compared to a specific mass or m/z value the result of that comparison is within some predetermined threshold level, such as, but not limited to, the resolution of the instrument used to make the comparison. Similarly, if two peaks are referred to as having the same mass or m/z value, these values are found to be the same within some predetermined threshold level, such as, but not limited to, the resolution of the instrument used to make the comparison.

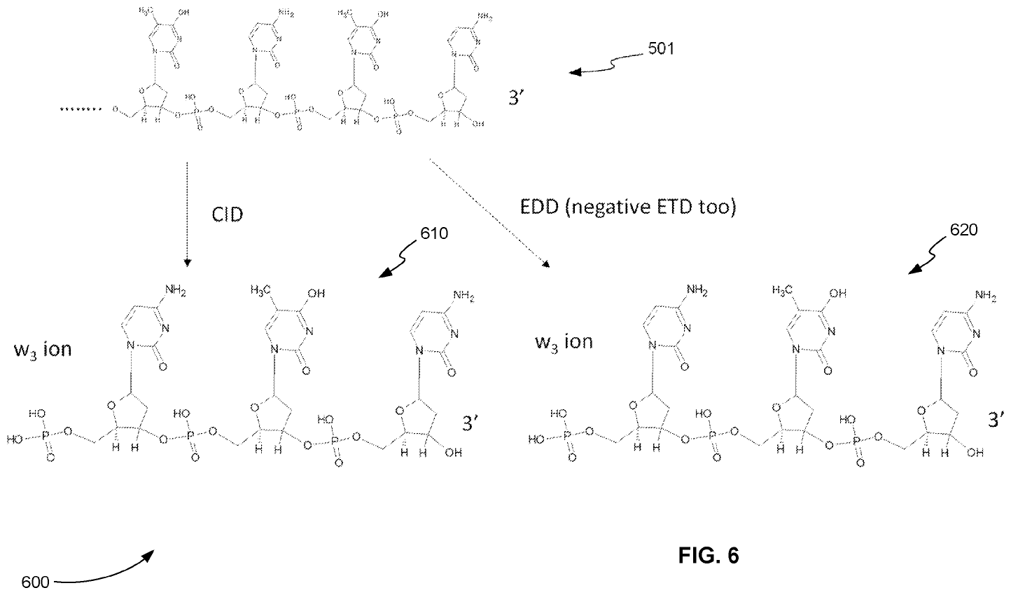

FIG. 6 is an exemplary diagram 600 showing that the fragmentation of the same nucleic acid by CID and EDD produces w ion series fragments that do not differ in mass, in accordance with various embodiments. FIG. 6 shows that the fragmentation of nucleic acid 501 by CID produces w ion series fragment or product ion 610. The fragmentation of nucleic acid 501 by EDD produces w ion series fragment or product ion 620. There is no difference in mass between w product ion 610 and w product ion 620. In other words, w product ion 610 and w product ion 620 are the same fragment. The existence of the same fragment in both spectra determines that w product ion 620 is a w ion series fragment candidate.

Next, each w ion series fragment candidate is subtracted from the precursor ion m/z value (z=−1) and the resultant m/z value is placed on the peak list ordered by m/z value.

Next, a virtual starting peak is added to the peak list ordered by m/z value. In various embodiments, the virtual starting peak has an m/z value of −81.981 (P₋₁O₋₃H₋₃). This is equivalent to a virtual a₀· fragment. This is the starting peak of de novo sequencing.

Next, a virtual ending peak is added to the peak list ordered by m/z value. In various embodiments, the virtual ending peak has an m/z value that is the precursor ion m/z value minus 19.018 (H₃O). This is equivalent to a virtual precursor ion with a· structure. This is the end point of de novo sequencing.

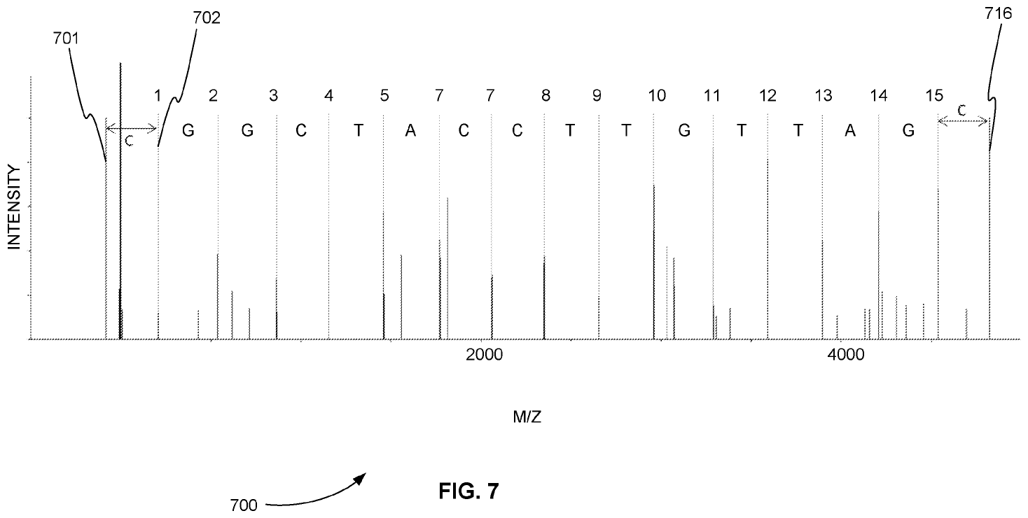

FIG. 7 is an exemplary plot 700 of the peak list ordered by m/z value, including a virtual starting peak, a· fragment candidates, and a virtual ending peak, in accordance with various embodiments. For example, in FIG. 7, peak 701 is the virtual starting peak and peak 716 is the virtual ending peak.

Next, de novo sequencing starts from the virtual starting peak of the peak list. A current m/z value or peak is set to the virtual starting peak. For example, in FIG. 7, the current peak is set to virtual starting peak 701.

Next, a next m/z value or peak of the peak list is determined that differs from the current peak by an a mass difference of 313.058 (C₁₀H₁₂N₅O₅P), 304.046 (C₁₀H₁₃N₂O₇P), 289.046 (C₉H₁₂N₃O₆P), or 329.053 (C₁₀H₁₂N₅O₆P), corresponding to nucleotides A, T, C, and G, respectively. For example, in FIG. 7, current peak 701 is found to differ from next peak 702 by a mass difference of 289.046, so nucleotide C of the sequence is found for the nucleic acid compound. In the case that such matched peak is not found in the peak list, combinations of four masses are examined as AA (=313.058+313.058), AT=(313.058+304.046), AC, AG, TT, TC, TG, CC, CG, GG, AAA, AAT, AAC, . . . )

Next, the current peak is set to the next peak that was found. For example, in FIG. 7, the current peak is set to next peak 702. The last two steps are then repeated until the next peak is found to be the virtual ending peak. For example, in FIG. 7, the previous step and this step are repeated until the next peak is found to be virtual ending peak 716.

As shown in FIG. 7, following this method, the 16-nucleotide sequence CGGCTACCTTGTTAGC is found for the nucleic acid compound from virtual starting peak 701 to virtual ending peak 716. The order of the found sequence is the sequence of the DNA from 5′ terminus to 3′ terminus.

In various embodiments, a conventional sequencing method is used to validate the sequence or find any missing nucleotides.

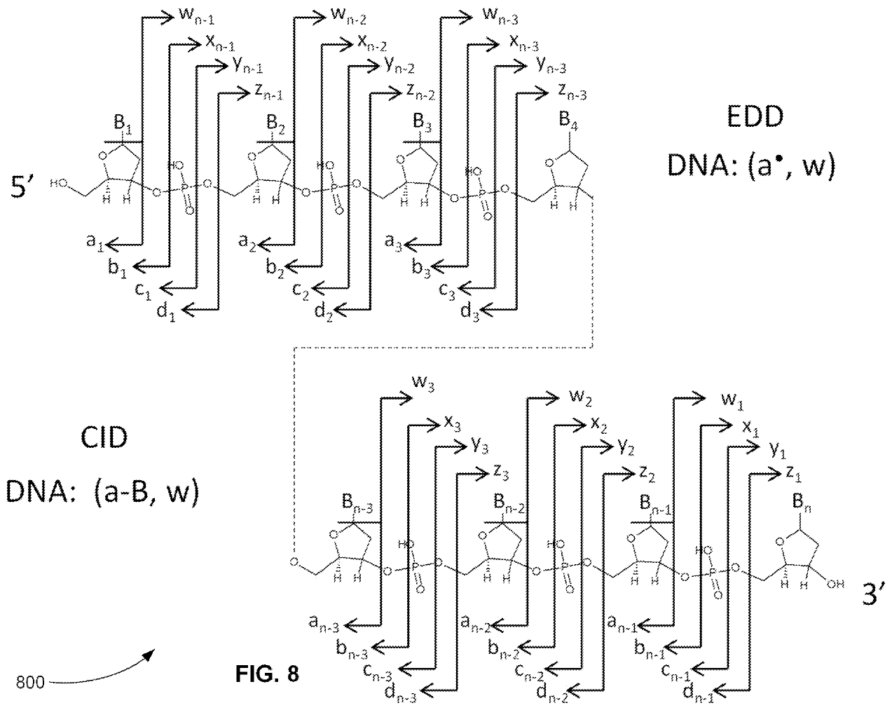

FIG. 8 is an exemplary diagram 800 showing the nomenclature of the different ion series fragments for a DNA compound and their relation to CID and EDD, in accordance with various embodiments. As described above, de novo sequencing starts at the a· equivalent equal to —PO₃H₂. De novo sequencing starts at the a· equivalent precursor ion mass equal to the precursor ion mass minus OH₃.

### System for Locating a Nucleotide During De Novo Sequencing

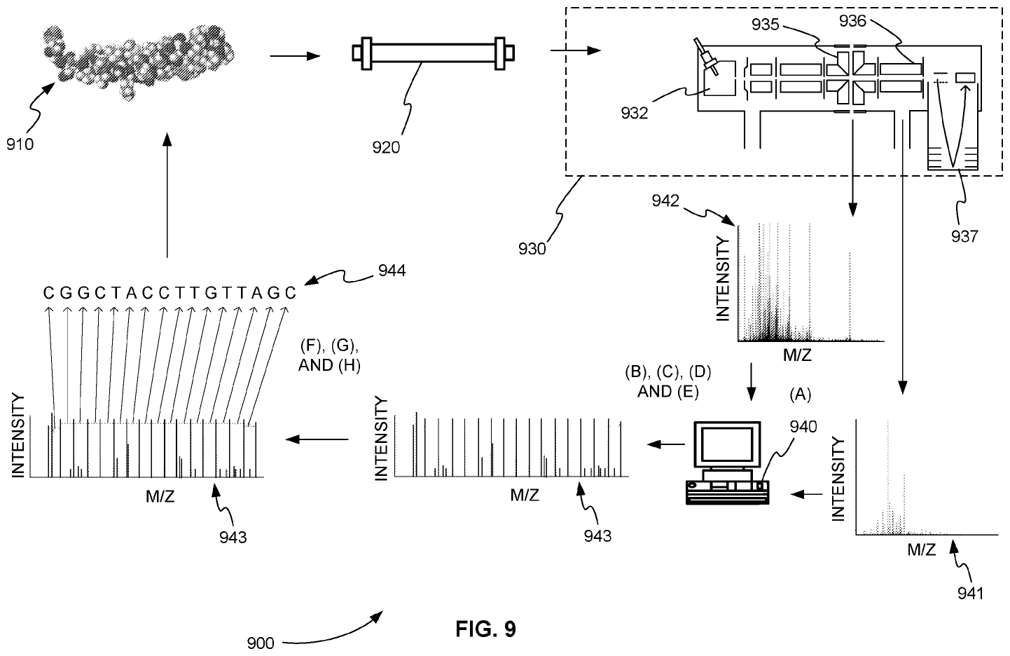

FIG. 9 is a schematic diagram 900 of a system for locating a nucleotide of a nucleic acid during de novo sequencing, in accordance with various embodiments. The system includes processor 940. Processor 940 can be, but is not limited to, a controller, a computer, a microprocessor, the computer system of FIG. 1, or any device capable of analyzing data. Processor 940 can also be any device capable of sending and receiving control signals and data.

In step (A), processor 940 receives first product ion mass spectrum 941 of nucleic acid 910 analyzed using a CID method. Processor 940 also receives second product ion mass spectrum 942 of nucleic acid 910 analyzed using a radical-induced dissociation method.

In optional step (B), processor 940 converts peak m/z values of first spectrum 941, peak m/z values of second spectrum 942, and an m/z value of a precursor ion of nucleic acid 910 to a single charge. In a preferred embodiment, this conversion is performed using charge state deconvolution. In various embodiments, the single charge is −1. The peaks in the spectra are converted to single charge in various embodiments. For this purpose, the charge state of each peak are identified from the carbon 13 isotope distribution, then the peak position and its peak distribution in the original horizontal scale (m/z scale) is theoretically (or mathematically) transferred to the single charge position with the single charge peak distribution. To calculate next candidates in de novo sequencing, the masses of the unit nucleotides are added to the single charged m/z value of the currently identified sequence from the started terminus. In the cases that the optional step (B) is not applied before de novo sequencing, original spectra are used. To calculate next candidates in de novo sequencing, the masses of the unit nucleotides are added to the single charged m/z value of the currently identified sequence from the started terminus. Required fragment types, such as a·, w, and a-B ions, are calculated. Then, the single charged m/z values are theoretically converted to m/z values with estimated multiple charges. A carbon 13 profile with m/z values matched to the calculated candidate m/z values are searched in the first and the second original spectra.

In a step (C), processor 940 determines a peak m/z value of first spectrum 941 that differs from a peak m/z value of second spectrum 942 by a mass difference of a compound within nucleic acid 910. This compound is one that the radical-induced dissociation method is known to be not able to dissociate and the CID method is known to be able to dissociate. The peak m/z value of second spectrum 942 then locates a nucleotide.

In various embodiments, the structure includes a phosphorus atom and an optionally substituted 5-membered ring containing an oxygen on the ring.

In various embodiments, the structure corresponds to one of the empirical formulas of FIG. 4 and with associated mass differences.

In various embodiments, step (C) is modified and additional steps are added to preform de novo sequencing. In step (C), processor 940 further determines each peak m/z value of first spectrum 941 that differs from a peak m/z value of second spectrum 942 by a mass of a compound within nucleic acid 910 and places the peak m/z value of second spectrum 942 on a peak list ordered by m/z value. This compound is one that the radical-induced dissociation method is known to not be able to dissociate and the CID method is known to be able to dissociate.

In various embodiments, in step (D), processor 940 subtracts each peak m/z value of first spectrum 941 that has the same peak m/z value as a peak of second spectrum 942 from the m/z value of the precursor ion of nucleic acid 910. Processor 940 also places the difference m/z value on peak list 943.

In various embodiments, in step (E), processor 940 adds a starting peak m/z value to peak list 943. Processor 940 also sets a current m/z value in peak list 943 to the starting peak m/z value.

In various embodiments, in step (F), processor 940 determines a next m/z value in peak list 943 that differs from the current m/z value by a first mass value of a first nucleotide, a second mass value of a second nucleotide, a third mass value of a third nucleotide, or a fourth mass value of a fourth nucleotide.

In various embodiments, in step (G), processor 940 stores a nucleotide corresponding to the difference between the next m/z value and the current m/z value as a nucleotide of sequence 944 of nucleic acid 910. Processor 940 also sets the current m/z value to the next m/z value.

In various embodiments, in step (H), processor 940 repeats steps (F) through (G) one or more times.

In various embodiments, in the case that a matched peak is not found in the peak list in step (F), combinations of two or more m/z mass values are examined (such as AA, AT, AC, AG, TT, TC, TG, CC, CG, GG, AAA, AAT, AAC . . . ). More specifically, in step (F), if a next m/z value is not found in peak list 943 that differs from the current m/z value by the first mass value, the second mass value, the third mass value, or the fourth mass value, then processor 940 determines a next m/z value from a combination of mass values. For example, processor 940 determines a next m/z value in peak list 943 that differs from the current m/z value by a combination of two or more mass values from the first mass value, the second mass value, the third mass value, and the fourth mass value and stores in step (G) nucleotides corresponding to the combination.

In various embodiments, the CID method includes a resonant CID method.

In various embodiments, the alternative radical-induced dissociation method includes a plasma EDD method.

In various embodiments, the alternative radical-induced dissociation method includes a beam-type negative electron-transfer dissociation (ETD) method.

In various embodiments, the alternative radical-induced dissociation method includes any UVPD, EPD, ECD, ETD, EDD, pEDD, or EED method.

In various embodiments, the structure includes C₅HO₅P⁻ and has a mass value of 179.0115.

In various embodiments, the starting peak m/z value comprises −81.981.

In various embodiments, processor 940 further, before step (F), calculates an ending peak m/z value by subtracting an end m/z value from the m/z value of the precursor ion of nucleic acid 910 and adding the ending peak m/z value to peak list 943.

In various embodiments, the end m/z value is the m/z value of the precursor ion converted to a single charge state minus 19.018 (H₃O).

In various embodiments, in step (H), processor 940 further repeats steps (F)-(G) until the current m/z value is the ending peak m/z value.

In various embodiments, the first nucleotide is an A nucleotide and the first mass value is 313.058, the second nucleotide is a T nucleotide and the second mass value is 304.046, the third nucleotide is a C nucleotide and the third mass value is 289.046, and the fourth nucleotide is a G nucleotide and the fourth mass value is 329.053.

In various embodiments, processor 940 further uses a conventional sequencing method to validate sequence 944 or to find any missing nucleotides in the sequence 944.

In various embodiments, the system of FIG. 9 further includes mass spectrometer 930. Ion source device 932 of mass spectrometer 930 ionizes the nucleic acid 910, producing an ion beam. Ion source device 932 is controlled by processor 940, for example. Ion source device 932 is shown as a component of mass spectrometer 930. In various alternative embodiments, ion source device 932 is a separate device. Ion source device 932 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 930 selects and fragments nucleic acid 910 and mass analyzes product ions of nucleic acid 910 from the ion beam. Mass spectrometer 930 further includes CID device 936, radical-induced dissociation device 935, and mass analyzer 937. Mass spectrometer 930 produces first spectrum 941 using CID device 936 and produces second spectrum 942 using radical-induced dissociation device 935.

In FIG. 9, mass analyzer 937 is shown as a time-of-flight (TOF) device. One of ordinary skill in the art can appreciate that mass analyzer 937 can be any type of mass analyzer including, but not limited to, a quadrupole, an ion trap, an orbitrap, or Fourier transform ion cyclotron resonance (FT-ICR) device.

In various embodiments, the system of FIG. 9 further includes a separation device 920 that separates nucleic acid 910 from a sample. As shown in FIG. 9, additional device 920 is an LC device. In various alternative embodiments, additional device 920 can be, but is not limited to, a gas chromatography (GC) device, capillary electrophoresis (CE) device, or an ion mobility spectrometry (IMS) device.

### Method for Locating a Nucleotide During De Novo Sequencing

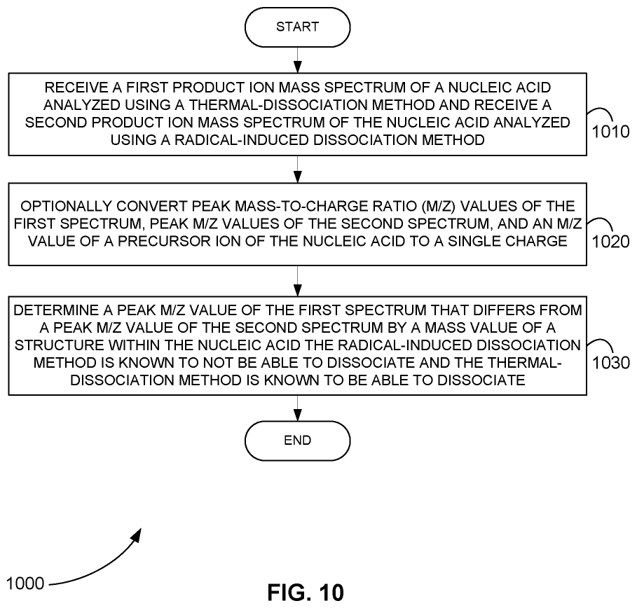

FIG. 10 is an exemplary flowchart showing a method 1000 for locating a nucleotide of a nucleic acid during de novo sequencing, in accordance with various embodiments.

In step 1010 of method 1000, a first product ion mass spectrum of a nucleic acid analyzed using a CID method is received. Also, a second product ion mass spectrum of the nucleic acid analyzed using a radical-induced dissociation method is received.

In an optional step 1020, peak m/z values of the first spectrum, peak m/z values of the second spectrum, and an m/z value of a precursor ion of the nucleic acid are converted to a single charge.

In step 1030, a peak m/z value of the first spectrum is determined that differs from a peak m/z value of the second spectrum by a mass value of a structure within the nucleic acid the radical-induced dissociation method is known to not be able to dissociate and the CID method is known to be able to dissociate.

### Computer Program Product for Locating a Nucleotide During De Novo Sequencing

In various embodiments, a computer program product includes a non-transitory tangible computer-readable storage medium whose contents include a program with instructions being executed on a processor so as to perform a method for locating a nucleotide of a nucleic acid during de novo sequencing. This method is performed by a system that includes one or more distinct software modules.

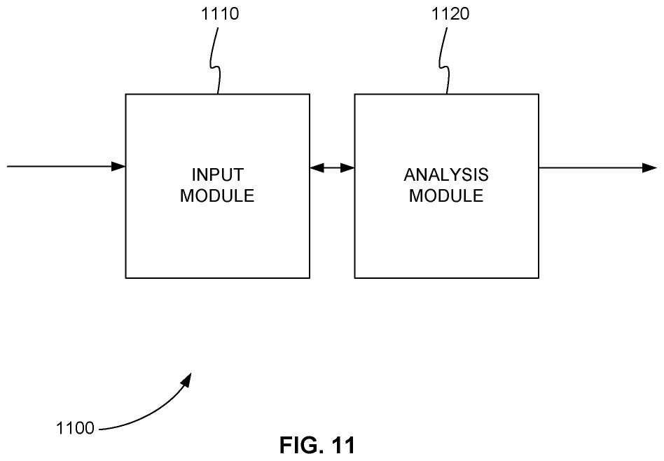

FIG. 11 is a schematic diagram of a system 1100 that includes one or more distinct software modules and that performs a method for locating a nucleotide of a nucleic acid during de novo sequencing, in accordance with various embodiments. System 1100 includes input module 1110 and analysis module 1120.

In step (A), input module 1110 receives a first product ion mass spectrum of a nucleic acid analyzed using a CID method. Input module 1110 also receives a second product ion mass spectrum of the nucleic acid analyzed using a radical-induced dissociation method.

In step (B), analysis module 1120 converts peak m/z values of the first spectrum, peak m/z values of the second spectrum, and an m/z value of a precursor ion of the nucleic acid to a single charge.

In step (C), analysis module 1120 determines a peak m/z value of the first spectrum that differs from a peak m/z value of the second spectrum by an m/z mass value of a structure within the nucleic acid the radical-induced dissociation method is known to not be able to dissociate and the CID method is known to be able to dissociate.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for locating a nucleotide of a nucleic acid during de novo sequencing, comprising:
(a) receiving a first product ion mass spectrum of a nucleic acid analyzed using a thermal-dissociation method and receiving a second product ion mass spectrum of the nucleic acid analyzed using a radical-induced dissociation method;
(b) optionally converting peak mass-to-charge ratio (m/z) values of the first spectrum, peak m/z values of the second spectrum, and an m/z value of a precursor ion of the nucleic acid to a single charge; and
(c) determining a peak m/z value of the first spectrum that differs from a peak m/z value of the second spectrum by a mass value of a structure within the nucleic acid in which the radical-induced dissociation method is known to not be able to dissociate and with which the thermal-dissociation method is known to be able to dissociate.

2. The method of claim 1, wherein the structure comprises a phosphorus atom and an optionally substituted 5-membered ring containing an oxygen on the ring.

3. The method of claim 1, wherein the structure corresponds to one of the following empirical formulas:
C₅HO₅P⁻, C₅H₈O₆P⁻, C₅H₉O₆PS⁻, C₅H₇FO₄PS⁻, C₆H₁₀O₅PS⁻, C₈H₁₄O₆PS⁻ or C₇H₁₂O₅PS⁻.

4. The method of claim 1, wherein the structure comprises C₅H₃O₅P and the mass value of the structure comprises 179.0115.

5. The method of claim 1, wherein step (c) further comprises
(c) determining each peak m/z value of the first spectrum that differs from a peak m/z value of the second spectrum by a mass value of a structure within the nucleic acid the radical-induced dissociation method is known to not be able to dissociate and the thermal-dissociation method is known to be able to dissociate and placing the peak m/z value of the second spectrum on a peak list ordered by m/z value.

6. The method of claim 1, further comprising
(d) subtracting each peak m/z value of the first spectrum that has the same peak m/z value as a peak of the second spectrum from the m/z value of the precursor ion of the nucleic acid and placing the difference m/z value on the peak list,
(e) adding a starting peak m/z value to the peak list and setting a current m/z value in the peak list to the starting peak m/z value,
(f) determining a next m/z value in the peak list that differs from the current m/z value by a first mass value of a first nucleotide, a second mass value of a second nucleotide, a third mass value of a third nucleotide, or a fourth mass value of a fourth nucleotide;
(g) storing a nucleotide corresponding to the difference between the next m/z value and the current m/z value as a nucleotide of a sequence of the nucleic acid and setting the current m/z value to the next m/z value, and
(h) repeating steps (f)-(g) one or more times to obtain the sequence.

7. The method of claim 6, further comprising in step (f), if a next m/z value is not found in the peak list that differs from the current m/z value by the first mass value, the second mass value, the third mass value, or the fourth mass value, then
determining a next m/z value in the peak list that differs from the current m/z value by a combination of two or more mass values from the first mass value, the second mass value, the third mass value, and the fourth mass value and storing in step (g) nucleotides corresponding to the combination.

8. The method of any claim 1, wherein the radical-induced dissociation method comprises a plasma electron detachment dissociation (pEDD) method or a beam-type negative electron-transfer dissociation (ETD) method.

9. The method claim 6, wherein the starting peak m/z value comprises −81.981.

10. The method of claim 6, further comprising, before step (f), calculating an ending peak m/z value by subtracting an end m/z value from the m/z value of the precursor ion of the nucleic acid and adding the ending peak m/z value to the peak list.

11. The method of claim 10, further comprising, in step (h), repeating steps (f)-(g) until the current m/z value comprises the ending peak m/z value.

12. The method of claim 6, wherein the first nucleotide comprises an A nucleotide and the first mass value comprises 313.058, the second nucleotide comprises a T nucleotide and the second mass value comprises 304.046, the third nucleotide comprises a C nucleotide and the third mass value comprises 289.046, and the fourth nucleotide comprises a G nucleotide and the fourth mass value comprises 329.053.

13. The method of claim 1, wherein the radical-induced dissociation method comprises one of an ultraviolet photodissociation (UVPD) method, an electron photodetachment dissociation (EPD) method, an electron capture dissociation (ECD) method, an electron transfer dissociation (ETD) method, an electron detachment dissociation (EDD) method, a plasma electron detachment dissociation (pEDD) method, or an electronic excitation dissociation (EED) method.

14. A computer program product, comprising a non-transitory tangible computer-readable storage medium whose contents cause a processor to perform a method for locating a nucleotide of a nucleic acid during de novo sequencing, comprising:
providing a system, wherein the system comprises one or more distinct software modules, and wherein the distinct software modules comprise an input module and an analysis module;
(a) receiving a first product ion mass spectrum of a nucleic acid analyzed using a thermal dissociation method and receiving a second product ion mass spectrum of the nucleic acid analyzed using a radical-induced dissociation method using the input module;
(b) optionally converting peak mass-to-charge ratio (m/z) values of the first spectrum, peak m/z values of the second spectrum, and an m/z value of a precursor ion of the nucleic acid to a single charge using the analysis module; and
(c) determining a peak m/z value of the first spectrum that differs from a peak m/z value of the second spectrum by a mass value of a structure within the nucleic acid the radical-induced dissociation method is known to not be able to dissociate and the CID method is known to be able to dissociate using the analysis module.

15. A system for locating a nucleotide of a nucleic acid during de novo sequencing, comprising:
a processor that
(a) receives a first product ion mass spectrum of a nucleic acid analyzed using a thermal dissociation method and receives a second product ion mass spectrum of the nucleic acid analyzed using a radical-induced dissociation method,
(b) converts peak mass-to-charge ratio (m/z) values of the first spectrum, peak m/z values of the second spectrum, and an m/z value of a precursor ion of the nucleic acid to a single charge, and
(c) determines a peak m/z value of the first spectrum that differs from a peak m/z value of the second spectrum by a mass value of a structure within the nucleic acid the radical-induced dissociation method is known not to be able to dissociate and the thermal-dissociation method is known to be able to dissociate.

16. The method of claim 1, wherein the thermal-dissociation method comprises CID or IRMPD.

17. The computer program product of claim 14, wherein the thermal-dissociation method comprises CID or IRMPD.

18. The system of claim 15, wherein the thermal-dissociation method comprises CID or IRMPD.
